(19) United States
(12) Patent Application Publication
(10) Pub. No.: US 2008/0310996 A1
(43) Pub. Date: Dec. 18, 2008

(54) Germicidal Floor System (GFS)

(76) Inventors: Darrick S. H. L. Kim, Schaumburg, IL (US); John Paoli, Crystal Lake, IL (US)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 12/101,494

(22) Filed: Apr. 11, 2008

Related U.S. Application Data
(60) Provisional application No. 60/911,616, filed on Apr. 13, 2007.

(51) Int. Cl. A61L 2/10

(52) U.S. Cl. 422/24; 422/186.3

(57) ABSTRACT

A device for cleaning feet, socks, or shoes comprises a housing, a target surface, and at least one ultraviolet light source. The target surface is supported by the housing and is adapted to be walked upon by an individual. The at least one ultraviolet light source is supported by the housing and adapted to direct ultraviolet light waves onto the target surface such that the ultraviolet light waves interact with the feet of the individual walking upon the target surface. The light waves kill or deactivate harmful germs or transmissible diseases disposed on the target surface, or carried by the feet or the socks of the individual, thereby preventing the harmful germs from spreading and causing harm to other individuals or animals.

### CROSS-REFERENCE TO RELATED APPLICATIONS

The priority benefit of U.S. Provisional Patent Application No. 60/911,616, filed Apr. 13, 2007, is claimed and the entire contents thereof are hereby incorporated herein by reference.

### FIELD OF THE INVENTION

The present invention generally relates to a device for cleaning feet and/or shoes worn on feet, and more particularly, to a device for killing and/or deactivating germs carried on feet and/or shoes worn on feet.

### BACKGROUND OF THE INVENTION

### Germicidal Ultraviolet Light

### What is Ultraviolet?

Ultraviolet light is part of the light spectrum, which is classified into three wavelength ranges:

UV-C, from 100 nanometers (nm) to 280 nm

UV-B, from 280 nm to 315 nm

UV-A, from 315 nm to 400 nm

### What is Germicidal Ultraviolet?

UV-C light is germicidal—i.e., it deactivates the DNA of bacteria, viruses and other pathogens and thus destroys their ability to multiply and cause disease. Specifically, UV-C light causes damage to the nucleic acid of microorganisms by making them form covalent bonds between certain adjacent bases in the DNA. The formation of such bonds prevents the DNA from being unzipped for replication, and the organism is unable to reproduce. In fact, when the organism tries to replicate, it dies. UVC has extremely low penetrating ability and does not penetrate past the dead-cell layers of the skin. UV will cause eye irritations or burns after prolonged exposure.

### What are the Beneficial Uses of Germicidal UV?

Ultraviolet technology is a non-chemical approach to disinfection. In this method of disinfection, nothing is added which makes this process simple, inexpensive and requires very low maintenance. Ultraviolet purifiers utilize germicidal lamps that are designed and calculated to produce a certain dosage of ultraviolet (usually at least 16,000 microwatt seconds per square centimeter but many units actually have a much higher dosage.) The principle of design is based on a product of time and intensity—you must have a certain amount of both for a successful design. Germicidal UV has been used in water disinfection systems for many years. As stated by the American Water Works Association, “ . . . UV light disinfection process does not use chemicals. Microorganisms, including bacteria, viruses, and algae, are inactivated within seconds of UV light disinfection . . . . Ultraviolet is effective in inactivating Cryptosporidium, while at the same time decreasing chlorinated disinfection by-products . . . . U.S. cities currently using or planning to use UV light water disinfection include: Seattle, Wash., New York City, Central Lake County, Illinois, Albany, New York, Tempe, Arizona . . . . ”

### Here are Just a Few of the Current Applications . . .

| Drinking Water | Food Processing |
|---|---|
| under sink installs & water vending | brewery & winery |
| machines | soft drinks, fruit drinks and juices |
| aircraft, boats & recreational vehicles | bottling facilities |
| water wells & water cisterns | diary processing |
| swimming pool & hot tubs | liquid sugars, sweeteners and |
| farms, ranches & trailer parks | edible oils |
| schools & hotels | water based lubricants |
| aquarium, hatcheries and nurseries | pure wash water |
| ice making | |
| Medical | Industries |
| pharmaceutical production | cosmetics and electronic |
| laboratories, hospitals and clinics | production |
| maternity labor and delivery areas | pond & lake reclamation |
| pathology labs, kidney dialysis | laundry water |
| animal husbandry | |

Numerous health studies have shown that germicidal ultraviolet light—UVC—is very effective against allergies, asthma, mold, mildew, fungi and any DNA based airborne viruses, bacteria and spores. UV light can play a great role in secondary allergy prevention especially where airborne allergens are concerned. It can reduce suffering from allergies. The UV air cleaners help control germs that make asthma worse. Ultraviolet Germicidal Irradiation—UVGI is the use of short UV waves, known as UVC, which has been proven to kill or inactivate tuberculosis in the indoor air. There are two ways in which UV lamps are used for indoor air quality control—upper-room air and in-duct ultraviolet germicidal irradiation. Many airborne pathogens, such as Anthrax and its spores, can be removed from the indoor air by the use of germicidal ultraviolet air purifiers.

Germicidal UV light (UVC) has been successfully utilized in hospitals and public buildings to inhibit microbial growth, spreading of infections and to increase the indoor air quality. UVC can also help against mold problems by rendering airborne mold particles and mold spores non-viable and even sterilizing surface mold colonies if they are directly irradiated with germicidal UV (UVC) light. Even the dead mold spores and mold particles can be allergenic or toxic so it is strongly recommended to always use germicidal UV light in conjunction with a HEPA filtration system.

How does Ultraviolet Light Work?

Germicidal ultraviolet lamp is a short wave low pressure mercury vapor tubes that produces ultraviolet wavelengths that are lethal to micro-organisms. Approximately 95% of the ultraviolet energy emitted is at the mercury resonance line of 254 nanometers. This wavelength is in the region of maximum germicidal effectiveness and is highly lethal to virus, bacteria and mold spores. Therefore, the genetic material of the micro-organism that is exposed to the germicidal ultraviolet light and is deactivated, which prevents them from reproducing and renders them harmless.

Germicidal UV light has been successfully utilized in hospitals and public buildings to inhibit microbial growth, spreading of infections and to increase indoor air quality. UVC can also help against mold problems for mold remediation and mold inhibition by rendering airborne mold particles and mold spores non-viable and even sterilizing surface mold colonies if they are directly irradiated with UVC light. These American Air & Water resources show mold/mildew irradiation dosages and indoor air testing results for mold and mold spores:

The following external resources present more information on mold/mildew cleaning and different mold remediation tips:

Please note that many variables take place in a real world environment that make actual calculating of the UV dosage very difficult (air flow, humidity, distance of microorganism to the UV light and time). However, it is proven that UV light will kill any DNA-based organism given enough UV dosage and that UV light breaks down DNA on a cumulative basis. The UV light helps to reduce incidences of inhaled pathogens for persons who reside or work in indoor environments.

The following are incident energies of germicidal ultraviolet radiation at 253.7 nanometers necessary to inhibit colony formation in microorganisms (90%) and for complete destruction:

| | Energy dosage of | |
|---|---|---|
| | Ultraviolet radiation | |
| | in μW/cm² needed | |
| Organisms: | for kill factor | |
| Bacteria | 90% | 100% |
| Bacillus anthracis - Anthrax | 4,520 | 8,700 |
| Bacillus anthracis spores - Anthrax | 24,320 | 46,200 |
| spores | | |
| Bacillus magaterium sp. (spores) | 2,730 | 5,200 |
| Bacillus magaterium sp. (veg.) | 1,300 | 2,500 |
| Bacillus paratyphusus | 3,200 | 6,100 |
| Bacillus subtilis spores | 11,600 | 22,000 |
| Bacillus subtilis | 5,800 | 11,000 |
| Clostridium tetani | 13,000 | 22,000 |
| Corynebacterium diphtheriae | 3,370 | 6,510 |
| Ebertelia typhosa | 2,140 | 4,100 |
| Escherichia coli | 3,000 | 6,600 |
| Leptospiracanicola - infectious | 3,150 | 6,000 |
| Jaundice | | |
| Microccocus candidus | 6,050 | 12,300 |
| Microccocus sphaeroides | 1,000 | 15,400 |
| Mycobacterium tuberculosis | 6,200 | 10,000 |
| Neisseria catarrhalis | 4,400 | 8,500 |
| Phytomonas tumefaciens | 4,400 | 8,000 |
| Proteus vulgaris | 3,000 | 6,600 |
| Pseudomonas aeruginosa | 5,500 | 10,500 |
| Pseudomonas fluorescens | 3,500 | 6,600 |
| Salmonella enteritidis | 4,000 | 7,600 |
| Salmonela paratyphi - Enteric fever | 3,200 | 6,100 |
| Salmonella typhosa - Typhoid fever | 2,150 | 4,100 |
| Salmonella typhimurium | 8,000 | 15,200 |
| Sarcina lutea | 19,700 | 26,400 |
| Serratia marcescens | 2,420 | 6,160 |
| Shigella dyseteriae - Dysentery | 2,200 | 4,200 |
| Shigella flexneri - Dysentery | 1,700 | 3,400 |
| Shigella paradysenteriae | 1,680 | 3,400 |
| Spirillum rubrum | 4,400 | 6,160 |
| Staphylococcus albus | 1,840 | 5,720 |
| Staphylococcus aerius | 2,600 | 6,600 |
| Staphylococcus hemolyticus | 2,160 | 5,500 |
| Staphylococcus lactis | 6,150 | 8,800 |
| Streptococcus viridans | 2,000 | 3,800 |
| Vibrio comma - Cholera | 3,375 | 6,500 |
| Molds | 90% | 100% |
| Aspergillius flavus | 60,000 | 99,000 |
| Aspergillius glaucus | 44,000 | 88,000 |
| Aspergillius niger | 132,000 | 330,000 |
| Mucor racemosus A | 17,000 | 35,200 |
| Mucor racemosus B | 17,000 | 35,200 |
| Oospora lactis | 5,000 | 11,000 |
| Penicillium expansum | 13,000 | 22,000 |
| Penicillium roqueforti | 13,000 | 26,400 |
| Penicillium digitatum | 44,000 | 88,000 |
| Rhisopus nigricans | 111,000 | 220,000 |
| Protozoa | 90% | 100% |
| Chlorella Vulgaris | 13,000 | 22,000 |
| Nematode Eggs | 45,000 | 92,000 |
| Paramecium | 11,000 | 20,000 |
| Virus | 90% | 100% |
| Bacteriopfage - E. Coli | 2,600 | 6,600 |
| Infectious Hepatitis | 5,800 | 8,000 |
| Influenza | 3,400 | 6,600 |
| Poliovirus - Poliomyelitis | 3,150 | 6,600 |
| Tobacco mosaic | 240,000 | 440,000 |
| Yeast | 90% | 100% |
| Brewers yeast | 3,300 | 6,600 |
| Common yeast cake | 6,000 | 13,200 |
| Saccharomyces carevisiae | 6,000 | 13,200 |
| Saccharomyces ellipsoideus | 6,000 | 13,200 |
| Saccharomyces spores | 8,000 | 17,600 |

### UV Tests

Tests conducted by Light Sources Inc—Orange, Conn. and verified by American Ultraviolet Company—Lebanon, Ind. revealed that American-Lights® produces 800 microwatts @ 1 foot with 534 feet per minute air flow @ 55° F. To compute time needed to sterilize germs in the following chart at 1 foot distance from the light, divide the dosage required by 800. Example: for 90% kill factor of Bacillus subtilis spores:11,600 divided by 800=14.5 seconds.

Microorganisms, including bacteria, viruses, and algae, are inactivated within seconds of UV light disinfection, but all are not equally sensitive. Generally, viruses and algae are more resistant to disinfection by UV light.

UV light is effective in inactivating Cryptosporidium, while at the same time decreasing chlorinated disinfection by-products.

UV disinfection is used in air and water purification, sewage treatment, protection of food and beverages, and many other disinfection and sterilization processes.

One major advantage of UV light disinfection is that it is capable of disinfecting water faster than chlorine, and without the need for retention tanks American Water Works Association

Mold and mold spores adversely affect indoor air quality (IAQ). Health conscious people or those suffering from the presence of mold in their homes need a certain level of knowledge and a lot of effort to clean and keep their environment healthy. Different types of mold—black mold, toxic mold, allergenic mold—are present all the time around us and in the air we breathe. In low levels, molds and mold spores are generally harmless but if their levels increase they can affect people; especially people with allergies, asthma and respiratory conditions or suppressed immune system.

### Athlete's Foot Fungi

A group of mold-like fungi called dermatophytes causes athlete's foot. These organisms sprout tendril-like extensions that infect the superficial layer of the skin. In response to this fungal growth, the basal layer of the skin produces more skin cells than usual. As these cells push to the surface, the skin becomes thick and scaly. Most often, the more the fungi spread, the more scales your skin produces, causing the ring of advancing infection to form.

“Athlete's foot causes dry, scaly skin, particularly between the toes, and can lead to splits in the heel. “As fingernails are one of the dirtiest parts of the body, I've seen serious secondary bacterial infections as a result of scratching and breaking the skin.”

Where Medicinal Treatments May be Used in Conjunction with Ultraviolet Light Treatment:

For mild conditions, your doctor may advise you to apply a prescription or over-the-counter (nonprescription) antifungal ointment, lotion, powder or spray. Most infections respond well to these topical agents, which include:

Terbinafine (Lamisil AT)

Clotrimazole (Lotrimin AF)

Miconazole (Micatin)

### Infectious Disease

An infectious disease is a clinically evident disease of humans or animals that damages or injures the host so as to impair host function, and results from the presence and activity of one or more pathogenic microbial agents, including viruses, bacteria, fungi, protozoa, multicellular parasites, and aberrant proteins known as prions. Transmission of an infectious disease may occur through several pathways; including through contact with infected individuals, by water, food, airborne inhalation, or through vector-borne spread.

A contagious disease (also called a communicable disease) is an infectious disease that is capable of being transmitted from one person or species to another. Contagious diseases are often spread through direct contact with an individual, contact with the bodily fluids of infected individuals, or with objects that the infected individual has contaminated.

The term infectivity describes the ability of an organism to enter, survive and multiply in the host, while the infectiousness of a disease indicates the comparative ease with which the disease is transmitted to other hosts. An infection however, is not synonymous with an infectious disease, as an infection may not cause clinical symptoms or impair host function.

### Classification

Among the almost infinite varieties of microorganisms, relatively few cause disease in otherwise healthy individuals. Infectious disease results from the interplay between those few pathogens and the defenses of the hosts they infect. The appearance and severity of disease resulting from any pathogen depends upon the ability of that pathogen to damage the host as well as the ability of the host to resist the pathogen. Infectious microorganisms, or microbes, are therefore classified as either primary pathogens or as opportunistic pathogens according to the status of host defenses.

Primary pathogens cause disease as a result of their presence or activity within the normal, healthy host, and their intrinsic virulence (the severity of the disease they cause) is, in part, a necessary consequence of their need to reproduce and spread. Many of the most common primary pathogens of humans only infect humans, however many serious diseases are caused by organisms acquired from the environment or which infect non-human hosts.

Organisms which cause an infectious disease in a host with depressed resistance are classified as opportunistic pathogens. Opportunistic disease may be caused by microbes that are ordinarily in contact with the host, such as bacteria or fungi in the gastrointestinal or the upper respiratory tract, and they may also result from (otherwise innocuous) microbes acquired from other hosts (as in Clostridium difficile enterocolitis) or from the environment as a result of traumatic introduction (as in surgical wound infections or compound fractures). An opportunistic disease requires impairment of host defenses, which may occur as a result of genetic defects (such as Chronic granulomatous disease), exposure to antimicrobial drugs or immunosuppressive chemicals (as might occur following poisoning or cancer chemotherapy), exposure to ionizing radiation, or as a result of an infectious disease with immunosuppressive activity (such as with measles, malaria or HIV disease). Primary pathogens may also cause more severe disease in a host with depressed resistance than would normally occur in an immunosufficient host.

One way of proving that a given disease is “infectious”, is to satisfy Koch's postulates (first proposed by Robert Koch), which demands that the infectious agent be identified only in patients and not in healthy controls, and that patients who contract the agent also develop the disease.

### Transmission

An infectious disease is transmitted from some source. Defining the means of transmission plays an important part in understanding the biology of an infectious agent, and in addressing the disease it causes. Transmission may occur through several different mechanisms. Respiratory diseases and meningitis are commonly acquired by contact with aerosolized droplets, spread by sneezing, coughing, talking or even singing. Gastrointestinal diseases are often acquired by ingesting contaminated food and water. Sexually transmitted diseases are acquired through contact with bodily fluids, generally as a result of sexual activity. Some infectious agents may be spread as a result of contact with a contaminated, inanimate object (known as a fomite), such as a coin passed from one person to another, while other diseases penetrate the skin directly.

Transmission of infectious diseases may also involve a “vector”. Vectors may be mechanical or biological. A mechanical vector picks up an infectious agent on the outside of its body and transmits it in a passive manner. An example of a mechanical vector is a housefly, which lands on cow dung, contaminating its appendages with bacteria from the feces, and then lands on food prior to consumption. The pathogen never enters the body of the fly.

### Methods of Control

Communicable diseases occur only when the causative agent comes into contact with a susceptible host in a suitable environment. Prevention and control efforts for communicable diseases may be directed to any of these three elements. Communicable diseases affect both individuals and communities, so control efforts may be directed at both. Treatment of persons with communicable diseases with antibiotics typically kills the agent and renders them noninfectious. Thus, treatment is also prevention. A simple way to prevent the occurrence of communicable diseases is to eliminate the infectious agent through, for example, cooking food, washing hands, and sterilizing surgical instruments between uses.

For most communicable diseases there is an interval between infection and occurrence of symptoms (the incubation period) in which the infectious agent is multiplying or developing. Some persons who are infected may never develop manifestations of the disease even though they may be capable of transmitting it (in apparent infection). Some persons may carry (and transmit) the agent over prolonged periods (carriers) whether or not they develop symptoms. Treatment during the incubation period may cure the infection, thereby preventing both disease and transmission.

The environment may be rendered less suitable for the occurrence of disease in a variety of ways. Reduction of crowding and appropriate ventilation can reduce the likelihood of droplet or airborne transmission.

### Diagnosis and Therapy

Diagnosis of infectious disease sometimes involves identifying an infectious agent either directly or indirectly. In practice most minor infectious diseases such as warts, cutaneous abscesses, respiratory infections and diarrheal diseases are diagnosed by their clinical presentation. Conclusions about the cause of the disease are based upon the likelihood that a patient came in contact with a particular agent, the presence of a microbe in a community, and other epidemiological considerations. Given sufficient effort, all known infectious agents can be specifically identified. The benefits of identification, however, are often greatly outweighed by the cost, as often there is no specific treatment, the cause is obvious, or the outcome of an infection is benign.

Specific identification of an infectious agent is usually only determined when such identification can aid in the treatment or prevention of the disease, or to advance knowledge of the course of an illness prior to the development of effective therapeutic or preventative measures.

### Clearance and Immunity

Infection with most pathogens does not result in death of the host and the offending organism is ultimately cleared after the symptoms of the disease have waned. This process requires immune mechanisms to kill or inactivate the inoculum of the pathogen. Specific acquired immunity against infectious diseases may be mediated by antibodies and/or T lymphocytes. Immunity mediated by these two factors may be manifested by:

a direct effect upon a pathogen, such as antibody-initiated complement-dependent bacteriolysis, opsonoization, phagocytosis and killing, as occurs for some bacteria,

neutralization of viruses so that these organisms cannot enter cells,

or by T lymphocytes which will kill a cell parasitized by a microorganism.

The immune response to a microorganism often causes symptoms such as a high fever and inflammation, and has the potential to be more devastating than direct damage caused by a microbe.

Resistance to infection (immunity) may be acquired following a disease, by asymptomatic carriage of the pathogen, by harboring an organism with a similar structure (crossreacting), or by vaccination. Knowledge of the protective antigens and specific acquired host immune factors is more complete for primary pathogens than for opportunistic pathogens.

### Impact of Communicable Diseases

Epidemics of “crowd” diseases such as measles and influenza resulted from person-to-person transmission.

Improvements in sanitation have dramatically reduced the burden of water-and food-borne diseases. Specific therapies such as antibiotics and anti-parasitic drugs have had a significant impact on deaths due to infectious diseases as well as having some impact on the occurrence of the diseases by shortening the period in which an infected person is infectious to others.

The most dramatic improvements have been seen in the United States and other developed nations. Although significant progress has also been made in developing nations, the World Health Report 2000 reports that 14 million deaths (25 percent of all deaths in the world in 1999) resulted from infectious diseases or their complications.

Much of the continuing toll of communicable diseases could be reduced by more effective use of existing vaccines and other tools for control of infectious diseases. However, new tools will be needed to bring about maximum control of some diseases. Because microorganisms are continually evolving, they may change enough so that prior experience (infection) with the infectious agent does not provide protection.

Mortality from Infectious Diseases

The World Health Organization collects information on global deaths by International Classification of Disease (ICD) code categories. The following table lists the top infectious disease killers which caused more than 100,000 deaths in 2002 (estimated). 1993 data is included for comparison.

The top three single agent/disease killers are HIV/AIDS, TB and malaria. While the number of deaths due to nearly every disease have decreased, deaths due to HIV/AIDS have increased fourfold. Childhood diseases include pertussis, poliomyelitis, diphtheria, measles and tetanus. Children also make up a large percentage of lower respiratory and diarrheal deaths.

### Emerging Diseases and Pandemics

In most cases, microorganisms live in harmony with their hosts. Such is the case for many tropical viruses and the insects, monkeys, or other animals in which they have lived and reproduced. Because the microbes and their hosts have co-evolved, the hosts gradually become resistant to the microorganisms. When a microbe jumps from a long-time animal host to a human being, it may cease to be a harmless parasite and become pathogenic.

With most new infectious diseases, some human action is involved, changing the environment so that an existing microbe can take up residence in a new niche. When that happens, a pathogen that had been confined to a remote habitat appears in a new or wider region, or a microbe that had infected only animals suddenly begins to cause human disease.

### Preventive Measures

Chemoprophylaxis. Chemoprophylaxis refers to the practice of giving anti-infective drugs to prevent occurrence of disease in individuals who are likely to be exposed to an infectious disease or who might have already been infected but have not developed disease.

Antibiotics and Resistance. Antibiotics are compounds that are produced by microorganisms that kill or inhibit the growth of other microorganisms. Those that kill bacteria are called bactericidal; those that prevent multiplication (and rely on the body's defense mechanisms to deal with the limited number of living organisms) are called bacteriostatic. Some antibiotics are effective against a limited number of microorganisms, others may have more widespread effect.

Because microorganisms are continually in a state of evolution, strains may evolve that are resistant to a particular antibiotic. In addition, resistance characteristics can be transferred from some microorganisms to others (this is particularly true of organisms that inhabit the gastrointestinal tract). The likelihood that resistance will develop is increased if antibiotics are used in an indiscriminate manner and in inadequate amounts (either in terms of individual dosage or in length of therapy). Antimicrobial resistance is a growing problem: organisms that once were exquisitely sensitive to a particular antibiotic may now have developed significant (or total) resistance to it. This necessitates either increasing the dose of the antibiotic administered (in the case of partial resistance) or developing totally new drugs to treat the infection (in the case of total resistance). A few microorganisms (such as enterococcus, an organism that lives in the intestinal tract and is particularly likely to cause infections in gravely ill patients with compromised immune systems) have developed such widespread resistance that it is a real challenge to treat them effectively, resulting in a need to develop even more antibiotics.

### Molds

Different types of mold—black mold, toxic mold, allergenic mold—are present all the time around us and in the air we breathe. In low levels, molds and mold spores are generally harmless but if their levels increase they can affect people; especially people with allergies, asthma and respiratory conditions or suppressed immune system.

Allergenic mold and mold spores are normally not dangerous to humans in low amounts, but they cause allergic or asthmatic symptoms. Generally, these types of mold can be relatively easy and safely cleaned and removed. Note that even dead mold spores or mold particles can trigger allergy symptoms or allergic reactions.

Mycotoxic mold and mold spores are those containing toxins in the cell wall. These types of mold can cause serious health problems in humans and animals. These molds range from short-term irritation to immunosuppression, to cancer and even death. If toxic molds are identified, it is suggested that you seek advice from an Industrial Hygienist or other mold professional for guidance. The average homeowner should NOT attempt the abatement of these types of mold.

Pathogenic mold is the type of mold that causes infections. Pathogenic molds can cause serious health effects in persons with suppressed immune systems, those taking chemotherapy, and those with HIV/AIDS, or autoimmunity disorders. If any pathogenic molds are identified, it is suggested that you seek advice from an Industrial Hygienist or other mold professional. The average homeowner should NOT attempt the abatement of this type of molds.

Hyphae & hyphal elements are single, unidentifiable fragments of mold. Although they might not be traceable to a specific mold species, these fragments can be responsible for allergic reactions in some people and may indicate previous or current growth. Ascospores and basidiospores are clusters of spores that may not be easily identified as a specific species, but may represent a mold problem in the property.

List of Infectious Diseases that May be of Concern:

### Viral Infectious Diseases

AIDS-AIDS Related Complex-Chickenpox (Varicella)-Common cold-Cytomegalovirus Infection-Dengue fever-Ebola haemorrhagic fever-Hand, foot and mouth disease-Hepatitis-Herpes simplex-Herpes zoster-HPV-Influenza (Flu)-Lassa fever-Measles-Marburg haemorrhagic fever-Infectious mononucleosis-Mumps-Poliomyelitis-Progressive multifocal leukencephalopathy-Rubella-Smallpox (Variola)-Viral encephalitis-Viral gastroenteritis-Viral meningitis-Viral pneumonia-West Nile disease-Yellow fever

### Bacterial Infectious Diseases

Anthrax-Bacterial Meningitis-Botulism-Brucellosis-Campylobacteriosis-Cat Scratch Disease-Cholera-Diphtheria-Epidemic Typhus-Gonorrhea-Impetigo-Legionellosis-Leprosy (Hansen's Disease)-Leptospirosis-Listeriosis-Melioidosis-MRSA infection-Plague-Pneumococcal pneumonia-Psiftacosis-Q fever-Rocky Mountain Spotted Fever (RMSF)-Salmonellosis-Scarlet Fever-Shigellosis-Syphilis-Tetanus-Trachoma-Tuberculosis-Tularemia-Typhoid Fever-Typhus; Urinary Tract Infections

### Parasitic Infectious Diseases

African trypanosomiasis-Amebiasis-Ascariasis-Babesiosis-Chagas Disease-Clonorchiasis-Cryptosporidiosis-Cysticercosis-Diphyllobothriasis-Dracunculiasis-Echinococcosis-Enterobiasis-Fascioliasis-Fasciolopsiasis-Filariasis-Free-living amebic infection-Giardiasis-Gnathostomiasis-Hymenolepiasis-Isosporiasis-Kala-azar-Leishmaniasis-Metagonimiasis-Myiasis-Onchocerciasis-Pediculosis-Pinworm Infection-Scabies-Taeniasis-Toxocariasis-Toxoplasmosis-Trichinellosis-Trichinosis-Trichuriasis-Trichomoniasis-Trypanosomiasis

### Fungal Infectious Diseases

Aspergillosis-Blastomycosis-Candidiasis-Coccidioidomycosis-Cryptococcosis-Histoplasmosis-Tinea pedis

Description of Infectious Diseases and Conditions that Directly Affect Foot

Foot infections can be difficult problems for physicians to treat due to the biomechanical complexities of the extremity and the underlying circumstances that cause the infections. Typically, they follow a traumatic event or tissue loss with contamination by foreign materials and/or colonization by bacteria. When a healthy patient or one without metabolic or peripheral vascular disease (PVD) presents with pedal infections, a traumatic process usually is involved. However, the more common presentation is that of a patient whose health is compromised with a metabolic or peripheral vascular defect that complicates optimum successful treatment.

Treatment strategies for foot infections have been changing and evolving as a result of pharmacologic and technical breakthroughs. Plastic and reconstructive techniques for limb salvage have altered the course of treatment for foot infections, with a goal towards functional restoration and a major decrease in amputation rates.

Physicians need to be aware of the many different types of foot infections that exist. Some foot infections are very simple, and others are quite complex. They are categorized into 3 groups including soft tissue, bone, and those associated with patients with diabetes. Foot infections in persons with diabetes can be unpredictable and are typically polymicrobial; therefore, they are discussed in their own venue. Soft tissue infections of the foot consist of any infectious process affecting the skin, subcutaneous tissue, adipose tissue, superficial or deep fascia, ligaments, tendons, tendon sheaths, joints, and/or joint capsules. Considering that there are more than 20 joints, 44 tendons, approximately 100 ligaments, 4 major compartments, and numerous fascial planes in the normal foot, one can easily recognize the potential for complex problems.

Many events can be responsible for these soft tissue infections. A description of soft tissue infections includes simple, moderate, and severe infections, which includes but is not limited to the immunocompromised patient, infections associated with PVD, emergency soft tissue infections, and infections associated with trauma.

Bone commonly is involved when any type of infectious process is present in the foot. Bone is predisposed to this because of its close proximity to the skin and lack of a thick, soft tissue, protective layer throughout most parts of the foot. This process with its diagnostic and treatment strategies is described in detail in this article.

Diabetes mellitus (DM) has been diagnosed in approximately 14 million US citizens. It can produce a complex clinical picture due to its involvement in numerous different organ systems. The combination of diabetic peripheral neuropathy and compromised distal vascularity act synergistically putting these patients at high risk for pedal complications. Individuals with diabetes tend to develop ulcerations in the feet, which often lead to infection of the soft tissue and bones.

### Cellulitis

Cellulitis is often the first sign of a soft tissue infection of the foot. In most cases, this marker represents an isolated localized skin infection but may represent a more severe process. Cellulitis usually originates from minor cuts and abrasions but also comes from more severe puncture wounds or trauma. Group A streptococci is the most common bacterial contaminant responsible for soft tissue infections, and Staphylococcus aureus is the second most common. Each is present in natural skin flora.

Initial treatment for simple cellulitis as a result of an abrasion in a host who is not immunocompromised includes oral antibiotics using first-generation cephalosporins, aminopenicillins, or quinolones. Group A streptococci is the most common pathogen and is usually susceptible to penicillin V and cephalexin. In more severe cases, oxacillin 2 g administered intravenously every 4 hours or cefazolin 1 g administered intravenously every 8 h can be used. Simple cellulitis usually responds well to antibiotics, rest, and elevation of the extremity. However, in more severe cases invasive treatment with debridement of necrotic tissue becomes necessary if septic embolization ensues.

### Paronychia

Paronychia is a more common soft tissue infection with inflammation of the periungual area adjacent to the nail grooves and borders. It can be initiated by a traumatic event such has dropping objects on the toes or having them stepped on; more often, paronychia results from an ingrown toenail (onychocryptosis). Underlying onychomycosis also can be a predisposing factor, which results in paronychia.

Initial treatment should include antibiotic therapy and warm soaks to the affected digit. Antibiotic therapy should be directed toward the offending pathogens, which are commonly skin flora. When onychocryptosis is the underlying etiology, that portion of the nail should be removed to address the soft tissue reaction. A partial nail avulsion involves removing the border of the ingrown nail. Chronic recurrent paronychias can be treated in a surgical manner both chemically and via local excision.

### Puncture Wounds

Puncture wounds occur more than 50% of the time on the planter surface of the foot with more than 90% of these involving penetration of a nail. Other objects in this category include wood, metal, plastic, glass, and animal and human bites. Puncture wounds have the potential to inoculate deep spaces of the foot, including bones, joints, tendons, and deep fascia, and serious complications can arise. Therefore, the depth of penetration is one of the most important factors in determining if a wound will resolve without complex intervention. Degree of infection can depend on location, type of penetrating object, retained foreign bodies from pieces breaking off, and penetration through shoes and socks.

The most common organisms implicated in penetrating wounds are S aureus, beta-hemolytic streptococci, and various anaerobic bacteria. Pasteurella multocida is typical in dog and cat bites or claw puncture wounds. Viridans streptococcus is responsible for most problems related to human bites. Pseudomonas aeruginosa is usually responsible for infection when the injury is due to object penetration through shoes and socks.

### Immunocompromise

Pedal infections in patients who are immunocompromised can be difficult to diagnose and treat due to comorbidities, which often alter the presentation and require treatment. This group of patients includes persons with HIV, systemic lupus erythematosus (SLE), rheumatoid arthritis (RA) and high-dose corticosteroid use, DM, and asplenia. These patients can have impaired host defenses and are at higher risk of acquiring infections.

### Human Immunodeficiency Virus

Patients with HIV are more susceptible to fungal and viral pedal infections. Tinea pedis and onychomycosis often are observed in this population. Human papilloma virus, manifesting itself as verruca plantaris, occurs at a higher rate than in the normal population. Fungal infections usually can be managed with a variety of antifungal creams. Verrucae can be more challenging to treat. The condition often requires several modalities for complete resolution. Common therapies include salicylic acid application, cryotherapy, blistering agents, and surgical excision.

### Systemic Lupus Erythematosus

Individuals with SLE are predisposed to infection due to their impaired humoral immunity and lowered T-lymphocyte-mediated immunity, which results from immunosuppressive therapy. Lupus flares can be mistaken for an infection and the differential diagnosis must be evaluated carefully. SLE skin and soft tissue infections most commonly are caused by S aureus and less commonly by group A streptococci. The absence of a true leukocytosis may create a confusing environment for the diagnostician.

### Rheumatoid Arthritis

Patients with systemic arthropathies such as RA are often on long-term corticosteroid therapy, which suppresses cell-mediated immunity; thus, a higher risk of infection results. Classic presentations include synovitis, pannus formation, and periarticular bone and cartilage destruction. These local manifestations of systemic disease can be confused with soft tissue infection. Approximately 20% of patients with RA have rheumatoid nodules. They frequently occur over pressure areas, which tend to rupture in the foot causing skin breakdown, erythema, and infection. Therefore, patients with RA presenting with possible pedal infections require a thorough workup to exclude the conditions discussed above.

### Diabetes

Persons with diabetes are truly compromised due to their impaired host defenses, decreased perfusion to the lower extremities, and diminished sensation in the feet secondary to peripheral neuropathy. This group of patients is very challenging to treat; treatment is covered in greater detail in Diabetic Foot Infections.

### Asplenia

The spleen is the primary site for immunoglobulin M (IgM) synthesis, which is the first early response of the body. This places patients with asplenia at higher risk for all infections. They are at a particular high risk for infection with encapsulated bacterial organisms. It is important to determine if they have joint implants or internal fixation in the feet because of the organisms' affinity for seeding these areas. For fractures requiring open reduction and external fixation (ORIF), prophylaxis against these pathogens is appropriate. Typical pedal infections in these patients are clinically indistinguishable from those in healthy hosts, except that they are often more severe.

Antibiotic coverage should be directed towards the encapsulated pathogens and consist of third- or fourth-generation cephalosporins. Imipenem should be used in the patient who is allergic to penicillin. If hardware is present and has become colonized, it should be removed for complete resolution of the infection.

### Peripheral Vascular Disease

The poorly perfused or ischemic foot is prone to more frequent and severe infections due to low oxygen tension. The inflammatory response to stress can be reduced. Decreased local perfusion, edema formation, and neutrophil infiltration are present. Small abrasions in these patients can become quite problematic because the lesions do not have sufficient blood supply to heal properly. Prolonged healing time leaves the patient more susceptible to infection and complications. Ischemic feet are also prone to ischemic-type ulcerations that have an extended healing time and an increased chance of infection.

### Necrotizing Fasciitis

Necrotizing fasciitis is characterized by widespread necrosis of fascia and deeper subcutaneous tissues, with initial sparing of skin and muscle. Eventually, skin involvement is noted, with cellulitis evolving into cutaneous gangrene. The most common underlying risk factor is being a patient with DM. One recent study by Elliott reported that foot ulcerations and infections associated with diabetes were the second most common cause of necrotizing fasciitis; thus, 15.2% of cases of necrotizing fasciitis are due to foot ulcerations and infections associated with diabetes.

Surgical wounds and infections resulting from intravenous drug abuse or “skin popping” also can lead to necrotizing infections. Aerobic streptococci are typical pathogens in addition to Bacteroides species, staphylococci, and enterococci, which all play a role in the infectious process. E coli and Proteus species are facultative anaerobic gram-negative rods that often are cultured from these wounds.

### Gas Gangrene

Gas gangrene or clostridial myonecrosis is considered a surgical emergency. It includes a rapid fulminating course, severe toxin-related systemic toxicity, a vast level of tissue destruction, and a high mortality rate. Rapid diagnosis and aggressive management are crucial with respect to limb preservation.

Six different species of Clostridia can be responsible for the soft tissue destruction; however, Clostridium perfringens is the most common. C perfringens produces 12 active tissue toxins responsible for the syndromes of gas gangrene. Clostridia organisms are saprophytes and are quite ubiquitous. Infections leading to gas gangrene require an opportunistic environment. Prerequisites include a wound, contamination with Clostridia organisms, and a depressed oxygen state at the site of inoculation. This accounts for the increased incidence of gangrene noted in patients with diabetes and patients with PVD. The decreased oxygen state also can be observed postoperatively from local edema and dressings.

### Chronic Osteomyelitis

Chronic osteomyelitis is defined as the presence of bone infection for more than 6 weeks. In the foot, it is most commonly observed in the diabetic population because their compromised immunity and vascular insufficiency predispose them to deep bone infections. Osteomyelitis also can be associated with open fractures, PVD, immunocompromised hosts, and improper treatment of the acute condition.

### Department of Homeland Security

Homeland security refers to governmental actions designed to prevent, detect, respond to, and recover from acts of terrorism, and also respond to natural disasters. The term became prominent in the United States following the Sep. 11, 2001 attacks; it had been used only in limited policy circles prior to these attacks. Before this time, such action had been classified as civil defense.

Homeland security is officially defined as “a concerted national effort to prevent terrorist attacks within the United States, reduce America's vulnerability to terrorism, and minimize the damage and recover from attacks that do occur,” according to the National Strategy for Homeland Security. Because the US Department of Homeland Security (DHS) includes the Federal Emergency Management Agency (FEMA) it has responsibility for preparedness, response and recovery to natural disasters as well.

Homeland security is generally used to refer to the broad national effort by all levels of government—federal, state, local and tribal—to protect the territory of the United States from hazards both internal and external as well as the Department of Homeland Security itself.

Homeland security is also usually used to connote the civilian aspect of this effort; “homeland defense” refers to its military component, led chiefly by the US Northern Command headquartered in Colorado Springs, Colo. Founded in 1921

The scope of homeland security includes:

Emergency preparedness and response (for both terrorism and natural disasters), including volunteer medical, police, Emergency Management and fire personnel;

Domestic intelligence activities, largely today within the FBI;

Critical infrastructure protection;

Border security, including both land and maritime borders;

Transportation security, including aviation and maritime transportation;

Biodefense;

Detection of nuclear and radiological materials;

Research on next-generation security technologies.

### In the United States

In the United States, the concept of “homeland security” extends and recombines responsibilities of much of the executive branch, including the Federal Bureau of Investigation (FBI), the National Guard, the Federal Emergency Management Agency (FEMA), the United States Coast Guard, the former Immigration and Naturalization Service (INS), the former U.S. Customs Service, the Secret Service, the Transportation Security Administration (TSA), and the Central Intelligence Agency (CIA). The George W. Bush administration has consolidated many of these activities under the United States Department of Homeland Security (DHS), a new cabinet department established as a result of the Homeland Security Act of 2002. However, much of the nation's homeland security activity remains outside of DHS; for example, the FBI and CIA are not part of the Department, and other agencies such as the Department of Defense and Department of Health and Human Services play a significant role in certain aspects of homeland security. Homeland security is coordinated at the White House by the Homeland Security Council, currently headed by Frances Townsend.

Airport security refers to the techniques and methods used in protecting airports and by extension aircraft from crime and terrorism.

Large numbers of people pass through airports every day. Such a large gathering of people presents a natural target for terrorism and other forms of crime due to the number of people located in a small area. Similarly, the high concentration of people on large airliners, the potential high lethality rate of attacks on aircraft, and the ability to use a hijacked airplane as a lethal weapon provide an alluring target for terrorism.

Airport security provides a first line of defense by attempting to stop would-be attackers from bringing weapons or bombs into the airport. If they can succeed in this, then the chances of these devices getting on to aircraft are greatly reduced. As such, airport security serves two purposes: To protect the airport from attacks and crime and to protect the aircraft from attack.

### Process and Equipment

Many past tragedies were the result of travelers allowed or able to carry either weapons or items that could be used as weapons on board aircraft so that they can hijack the plane. Travelers are quickly screened by a metal detector. More advanced explosive detection machines are being used in screening passengers. Baggage must be screened to prevent the carrying of bombs aboard an aircraft. X-ray machines are often used to speed this process. Explosive detection machines can also used for both carry on and checked baggage. These detect volatile compounds given off from explosives using a kind of gas chromatography. A recent development is the use of X-ray back scatter scanners to detect hidden weapons and explosives on passengers. These devices, which use Compton scattering, require that the passenger stand close to a flat panel and produce a high resolution image.

Generally people are screened through airport security into the concourses, where the gates are all located. This area is often called a secure or sterile area. Passengers are discharged from airliners into the sterile area so that they usually will not have to be rescreened if boarding a domestic flight; however they are still subject to search at any time. For those airports that have sit down eating establishments, a common feature is that they will use plastic cutlery and paper cups rather than metal cutlery and glasses made out of glass, lest they be used as a weapon. Traditionally, non-passengers were allowed on the concourses to meet arriving friends or relatives at their gates, but this is no longer allowed in the interest of security in the United States.

In some countries, specially trained individuals may engage passengers in a conversation to detect threats rather than solely relying on equipment to find threats.

### Notable Incidents

The single deadliest airline catastrophe resulting from the failure of airport security to detect an onboard bomb was Air India Flight 182, which killed 329 people.

Another notable failure was the 1994 bombing of Philippine Airlines Flight 434, which turned out to be a test run for a planned terrorist attack called Operation Bojinka. The explosion was small, killing one person, and the plane made an emergency landing. Operation Bojinka was discovered and foiled by Manila police in 1995.

On May 30, 1972 three members of the Japanese Red Army undertook a terrorist attack, popularity called the Lod Airport massacre, at the Lod Airport, now known as the Ben Gurion International Airport, in Tel Aviv. Firing indiscriminately with automatic firearms and throwing grenades, they managed to kill 24 people and injure 78 others before being neutralized (one of them through suicide). One of the three terrorists, Kozo Okamoto, survived the incident.

The Rome and Vienna airport attacks in December 1985 were two more instances of airport security failures. The attacks left 20 people dead when gunmen threw grenades and opened fire on travelers at El Al ticket counters.

On Aug. 10, 2006, security at airports in the United Kingdom and the United States was raised significantly due to the uncovering by British authorities of a terror plot aimed at detonating liquid explosives on flights originating from these countries. This is also notable as it was the first time the US Terror Alert Level ever reached Red. This lead to tighter restrictions to carry liquid materials in hand luggage across the EU.

### United States

Prior to the 1970s American airports had minimal security arrangements to prevent aircraft hijackings. Screening measures were introduced starting in the late 1960s after several high-profile hijackings.

Sky marshals were introduced in 1970 but there were insufficient numbers to protect every flight and hijackings continued to take place. Consequently in late 1972, the FAA required that all airlines begin screening passengers and their carry-on baggage by Jan. 5, 1973. This screening was generally contracted to private security companies. Private companies would bid on these contracts, with the lowest bid usually being the winning one. The airline that had operational control of the departure concourse controlled by a given checkpoint would hold that contract. Although an airline would control the operation of a checkpoint, oversight authority was held by the FAA. C.F.R. Title 14 restrictions did not permit a relevant airport authority to exercise any oversight over checkpoint operations.

The Sep. 11, 2001 attacks prompted even tougher regulations, such as limiting the number of and types of items passengers could carry on board aircraft and requiring increased screening for passengers who fail to present a government issued photo ID.

The Aviation and Transportation Security Act generally required that by Nov. 19, 2002 all passenger screening must be conducted by Federal employees. As a result, passenger and baggage screening is now provided by the Transportation Security Administration (TSA), part of the Department of Homeland Security. Provisions to improve the technology for detecting explosives were included in the Terrorism Prevention Act of 2004.

### The Airport Screening Experience

Everyone who travels by air goes through airport security checkpoints. These checkpoints are operated by Transportation Security Officers from the Transportation Security Administration.

The checkpoints are there to make sure that terrorists can't bring anything aboard the plane that would enable them to take it over or destroy it. These are called “prohibited items” and cannot be brought to a checkpoint, into the secure area of an airport, or aboard an aircraft.

Not only do all passengers go through checkpoints, their checked baggage is also screened. This may happen out of passengers' view depending on the physical configuration at each airport.

Disposable booties or slippers may be worn through the checkpoint to help protect feet, but they must be disposed of prior to leaving the screening area.

Everyone is required to remove their shoes before entering the walk-through metal detector. All types of footwear must be placed on the X-ray machine to be screened.

Due to the Homeland Security threat level being raised for the U.S. aviation sector worldwide this is critical to protect the world's travelers who transit by air to and from the United States.

TSA Security Officers will ask the passenger to remove his/her shoes before entering the metal detector based on the fact that many types of footwear can be used to carry prohibited items. If a person refuses to remove their shoes when asked by a Security Officer, that person will not be able to board the flight.

### Why We Screen Shoes

TSA instituted mandatory shoe screening as an additional security measure when the threat level for the aviation sector went to Orange, or high on Thursday, Aug. 10, 2006. Screening shoes by x-ray is an effective method of identifying any type of anomalies including explosives. Highly trained transportation security officers can see if a shoe has been tampered with when they view it on the X-ray equipment. By requiring all passengers to remove shoes for x-ray screening we increase both security and efficiency at the checkpoint.

Today improvised explosive devices are the number one threat that we guard against. More than 31,000 of our TSA have received rigorous training in IED detection and are required to complete four hours a month of recurrent training to detect all forms of explosives. Training and technology are two layers in our systems approach to security in the airport. Other layers include: intelligence, behavior observation technique, random canine team searches, federal air marshals, federal flight deck officers and additional security measures both visible and invisible to the public. Each one of these layers alone is capable of stopping a terrorist attack. In combination their security value is multiplied, creating a much stronger, formidable system.

### Other Applications

### 1. Germs in Gym

Everyone must agree that one of the most germ-infested places in sports facility is the floor.

Sports facility and shared equipment has been linked to the spread of a new type of MRSA (methicillin-resistant Staphylococcus aureus) in athletic and sports teams.

“Community-acquired strains of MRSA are quite different from the hospital type,” says Dr Sally Bloomfield of the International Scientific Forum on Home Hygiene in London.

“They have acquired the ability to produce a potent tissue toxin called Panton-Valentine Leukocidin (PVL), which can lead to skin and soft tissue infections, including flesh-eating forms.

“These bacteria can infect the young and healthy. Transmission via close contact, sharing towels and sports equipment is a significant risk factor.”

More than half of us carry the ‘old type’ of Staphylococcus aureus on our skin. Anyone with broken skin—cuts, wounds or abrasions—is at risk of contracting a staph infection from someone else.

Threadworm parasites and infections such as diarrhoea and tummy upsets can be transmitted through contact with dirty gym floor.

The average gym mat contains 100,000 bacteria per square centimetre, and some contain faecal bacteria (that could causes tummy upsets).

Podiatrists also blame dirty exercise mats and floors of sports facility for a rise in athlete's foot and veruccas.

“Fungus thrives in moist environments, so if they are not kept spotless, mats can be the perfect breeding ground.

“If this goes unchecked, it can lead to toenail fungus characterised by blackening of the nails.”

Every workout places is of the risk of the new strains of community-acquired MRSA.

Showers, saunas and locker rooms are rife with viruses and fungal diseases that can cause athlete's foot, veruccas and onychomycosis (a fungal infection of toenails or fingernails).

“It can get worse without treatment and becomes red, inflamed and itchy, particularly when your feet are warm. “Even if you wash and dry your feet, the minute you start sweating, the bacteria eats the sweat and excretes waste, giving off a strong odour.”

Working out in unsupportive and sweaty shoes carries risk of injury and fungal infection. There are 100 times more yeasts and moulds in old boots and trainers than in a flushed toilet bowl.

Moist conditions make sports shoes a perfect breeding ground for fungi,

Shoes worn outside, where there is animal and bird feces, can carry bacteria that other people then pick up on their shoes and bags.

There could be more than 100 million bacteria per square inch on floors.

“If you get those germs on your fingers, then rub your eyes, nose or mouth, you could get diarrhoea.” Studies also show that changing room benches are not much cleaner.

### 2. Pedicures Salons

### Mycobacterium Fortuitum:

In 2000, an outbreak of Mycobacterium fortuitum furunculosis affected customers using whirlpool footbaths at a nail salon.

M. fortuitum is a relatively harmless bacterium when it is present in small numbers. In order to understand Mycobacterium fortuitum one needs to know what it is, its pathology, and how it is treated when an outbreak occurs.

Mycobacterium fortuitum is a type of rapidly growing mycobacteria (RGM) and is classified as (an atypical) mycobacteria, which encompasses all mycobacteria outside of Mycobacterium tuberculosis. Mycobacteria are unicellular, Gram-positive cells with a thick hydrophobic cell wall that aids in the prevention of desiccation. The growth of bacteria depends on temperature and a variety of nutrients.

The pathology of Mycobacterium fortuitum is somewhat simplistic. The bacterium is not spread from person to person. Rather, humans acquire it from the environment. Once humans have been infected with M. fortuitum, it can become harmful. According to Eduardo Nagore, M. fortuitum causes soft-tissue and skeletal infections due to direct inoculation of contaminated materials via injections, surgery, and penetrating trauma. To add, these contaminated materials usually pick up M. fortuitum through unintentional contact with water. The strength of ones immune system can also influence how at risk a person is to getting a disease due to a M. fortuitum infection. E. Leoni noted that people with suppressed immune systems have a greater chance of developing an infection. Consequently, everyone is at risk to some degree of contracting an infection due to M. fortuitum, because it is present in water, water sources, and soil, which everyone comes in contact with on a daily basis.

One of the largest outbreaks of Mycobacterium fortuitum took place in a California nail salon. In September 2000, a dermatologist in Northern California reported to the state health department that four of her patients had persistent boils on their legs below the knee and a common factor between all of them was that they received pedicures from the same nail salon. Dr. Kevin L. Winthrop and his colleagues investigated the situation and performed the first case study on the outbreak of M. fortuitum in a nail salon. They found that 110 customers had furunculosis, which caused boils on the nail salons clients shins and of those people, 34 of them tested positive for M. fortuitum. All of the women had the same symptoms for the bacterium. After getting pedicures, small sores developed on their lower legs and after several weeks or months, they became large, tender, boils with some even progressing to skin ulcers. Additionally, each infected client had between one and thirty-seven boils on each leg.

The Federal Centers for Disease Control concluded that the size of the boils ranged from as small as a nickel to as large as a half dollar. With all this information, the next step was to determine the exact source of this outbreak. Through a series of tests it was discovered that a single strain of M. fortuitum was responsible for the follicular infections, which caused the boils. The bacterium got into the salon through a water source. It was concluded that the bacterium entered the salon through the tap water, grew in the accumulated debris of hair, skin, and nails behind the footbath inlet screens, which were never cleaned, and multiplied rapidly due to the warm, nutrient rich environment. In fact, this incident most likely could have been avoided had the organic debris, behind the screens, been cleaned out.

In the case of furunculosis resulting from the outbreak of M. fortuitum at the nail salon, some of the customers were left with extensive scaring due to the boils after being treated with antibiotics to kill the bacterium. The removal of the scars would require skin grafts or laser surgery. As one can see, infections caused by M. fortuitum are treatable, but it can be a long, agonizing process.

### 3. Germs at Home

Shoes worn outside of a house carries in as much if not more germs as those worn in an athletic facility. In particular when a household also raises a pet dog or a cat, the risk of exposure to harmful germs rises as these pets venture in and out of the house. No one knows what these pets may have stepped on. It is not difficult to imagine when an individual walks barefoot or even in socks in the house is at risk of contracting highly transmissible disease causing germs through contact on the floor. Thus it may be a good suggestion to sanitize one's feet now and then from harmful germs that may have gotten on from the floor now and then, in particular when the individual is at risk of infection due to lowered immune conditions such as diabetes, AIDs, lupus, scratch or punctured wounds on the feet etc. . . .

### SUMMARY OF THE DISCLOSURE

The present disclosure provides a device for cleaning feet, socks, or shoes comprises a housing, a target surface, and at least one ultraviolet light source. The target surface is supported by the housing and is adapted to be walked upon by an individual. The at least one ultraviolet light source is supported by the housing and adapted to direct ultraviolet light waves onto the target surface such that the ultraviolet light waves interact with the feet of the individual walking upon the target surface. The light waves kill or deactivate harmful germs or transmissible diseases disposed on the target surface, or carried by the feet or the socks of the individual, thereby preventing the harmful germs from spreading and causing harm to other individuals or animals.

In one embodiment, the device can further comprise a means for moving air through the target surface and adjacent the at least one ultraviolet light source to kill or deactivate air born germs or transmissible diseases released in the area of the target surface.

So embodied, the device could further comprise a germicidal filter disposed adjacent to the target surface such that at least some of the air circulated by the air circulation means travels through the germicidal filter.

In at least one embodiment, the target surface can comprise a porous surface and the means for circulating air through the target surface can comprise a fan for drawing air downward through the target surface.

In another embodiment, the device can further comprise a means for emitting a germicidal chemical in the area of the target surface to kill or deactivate harmful germs or transmissible diseases.

In one form, the germicidal chemical can comprise an antibiotic chemical.

In one embodiment, the ultraviolet light waves generated by the ultraviolet light sources can comprise at least one of UV-C light waves, UV-B light waves, and UV-A light waves.

In one embodiment, the housing can comprise a pair of channels disposed on opposite sides of the target surface such that the at least one ultraviolet light source and a light reflector can be disposed within at least one of the channels.

So embodied, the channels can be shaped and configured to include elongated openings disposed adjacent to the target surface for directing the ultraviolet light waves toward the target surface.

In at least one embodiment, the target surface can comprise a removable grate to enable the removal of trapped debris from beneath the target surface.

In a still further alternative embodiment, the present disclosure provides a device including a housing, target surface, a first ultraviolet light source, and a second ultraviolet light source. The housing defines a base plate, and first and second channels extending along opposite sides of the base plate. The target surface has discrete dimensions and is supported by the base plate of the housing and adapted to be walked upon by an individual. The first ultraviolet light source and a light reflector are supported by the housing within the first channel. The second ultraviolet light source and a light reflector are supported by the housing within the second channel.

So configured, the first and second channels of the housing are shaped and configured with the light reflector to reflect ultraviolet light waves generated by the first and second ultraviolet light sources onto the target surface. The ultraviolet light waves thereby interact with the feet of the individual walking upon the target surface to kill or deactivate harmful germs or transmissible diseases disposed on the target surface, or carried by the feet or socks of the individual to prevent the harmful germs or transmissible diseases from spreading and causing harm to other individuals or animals.

In one embodiment, the first and second channels of the housing can define first and second elongated openings extending the length of the target surface.

In another embodiment, the device can further comprise a means for moving air through the target surface and into the first and second channels adjacent to the first and second ultraviolet light sources to kill or deactivate air born germs or transmissible diseases released in the area of the target surface.

So embodied, the device can further comprise a germicidal filter disposed adjacent to the target surface such that at least some of the air circulated by the air circulation means travels through the germicidal filter.

In one embodiment, the target surface of the device can comprise a porous surface and the means for circulating air through the target surface comprises a fan for drawing air downward through the target surface.

In another embodiment, the device can further comprise a means for emitting a germicidal chemical in the area of the target surface to kill or deactivate harmful germs.

In one form, the germicidal chemical can comprise an antibiotic chemical.

In one embodiment, the ultraviolet light waves can comprise at least one of UV-C light waves, UV-B light waves, and UV-A light waves.

In another embodiment, the target surface can comprise a removable grate supported by the base plate of the housing to enable the removal of trapped debris from beneath the target surface.

Thus, the present disclosure also describes a method of killing or deactivating harmful germs or transmissible diseases disposed on a target surface of a cleaning device, or carried by the feet or socks of an individual walking on the target surface.

One embodiment of such a method can first comprise generating ultraviolet light waves with one or more ultraviolet light sources disposed within channels disposed on opposing sides of the target surface of the cleaning device.

The method can further include one or more of the following steps:

(1) deflecting the ultraviolet light waves out of the channels and toward the target surface such that the ultraviolet light waves interact with at least one of the target surface, the individuals feet walking across the target surface, and the socks worn by the individual walking across the target surface to kill or deactivate harmful germs or transmissible diseases disposed on the target surface or carried by the feet or socks of the individual;

(2) circulating air through the target surface and toward the one or more ultraviolet light sources disposed within the channels to kill or deactivate air born germs or transmissible diseases released in the area of the target surface; and/or

(3) emitting a germicidal chemical in the area of the target surface to kill or deactivate harmful germs or transmissible diseases.

### BRIEF DESCRIPTION OF THE DRAWINGS

### The Design:

Grid floor with ultraviolet light shining from the floor where people can walk on or simply stand on for a short duration, a few seconds to several seconds, depending on the desired needs of each individual.

The attached drawings are representative ideas and designs of the germicidal floor system. The system is designed to protect individuals from direct exposure of UV lights to their eyes while effectively covering areas exposed to foot traffic. As one can clearly see, the devices can be of many different designs.

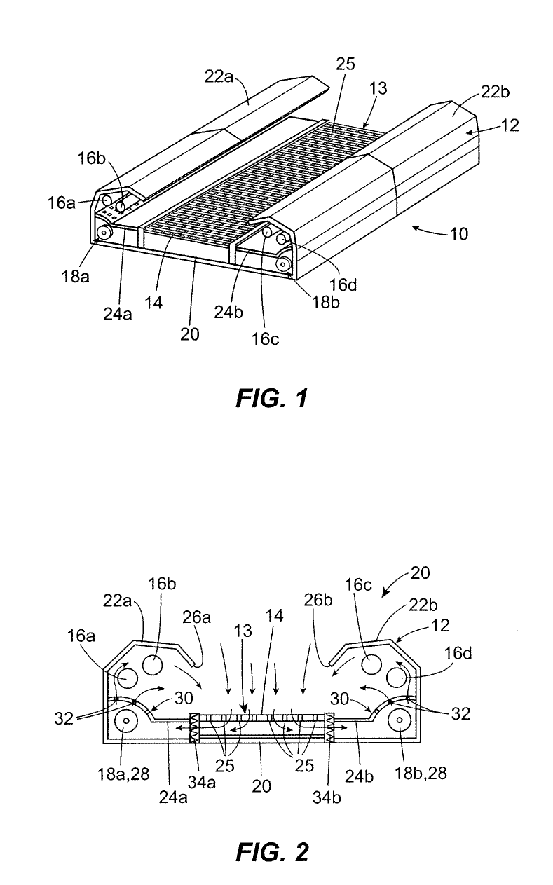

FIG. 1 is a perspective view of one embodiment of a germicidal floor system constructed according to the principles of the present invention;

FIG. 2 is a cross-sections end view of the germicidal floor system of FIG. 1 with arrows showing airflow;

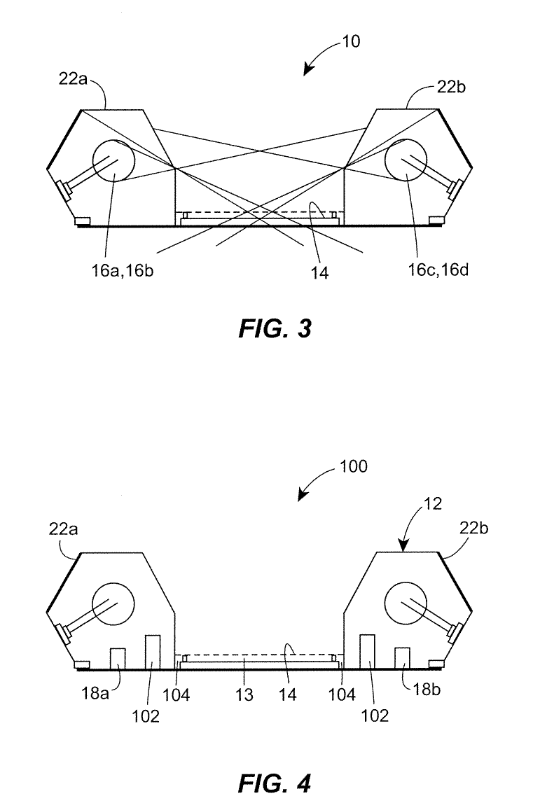

FIG. 3 is a schematic end view of the germicidal floor system of FIGS. 1 and 2 showing light beam projections from ultraviolet light sources;

FIG. 4 is a schematic end view of an alternative embodiment of a germicidal floor system constructed according to the principles of the present invention and including a drainage system for draining flow of germicidal fluids;

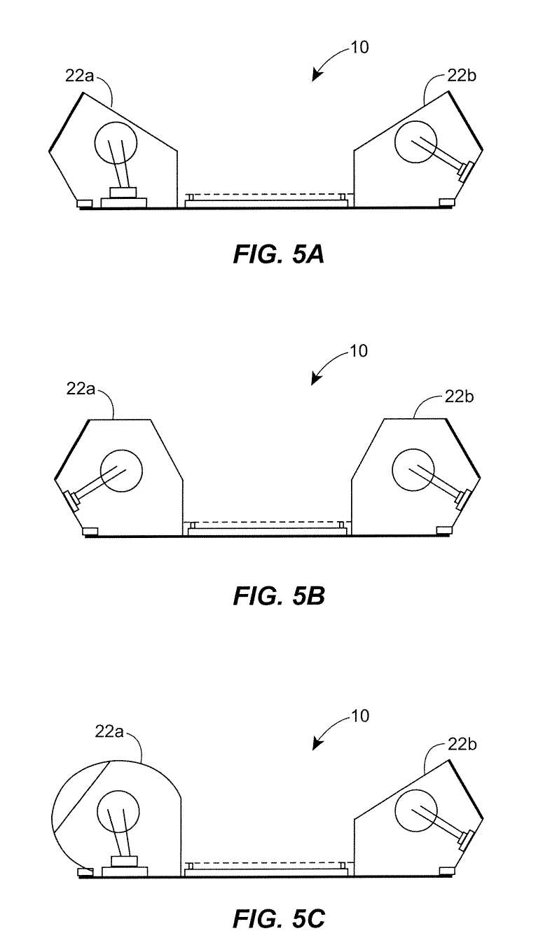

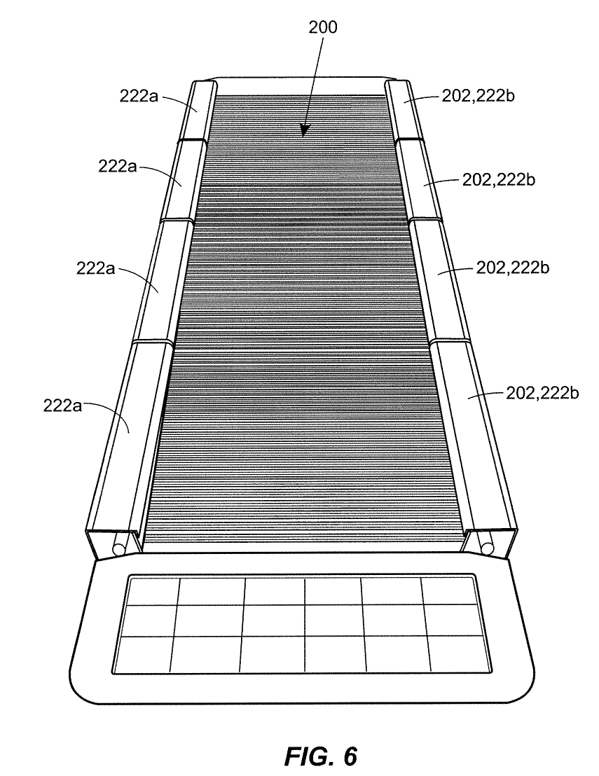

FIGS. 5A-5C are schematic end views of further alternative embodiments of germicidal floor systems constructed in accordance with the principles of the present invention;

FIG. 6 is a perspective view of a prototype germicidal floor system constructed in accordance with the principles of the present invention and including a plurality of prototype germicidal floor system components; and

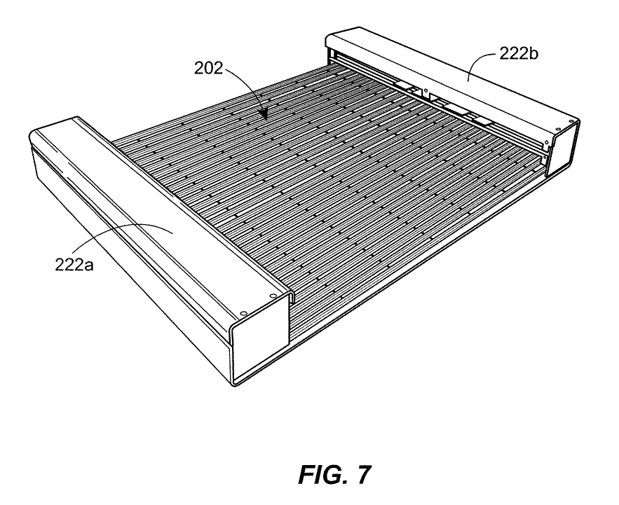

FIG. 7 is a perspective view of one of the prototype germicidal floor system components of FIG. 6.

### GENERAL DESCRIPTION OF THE INVENTION

This invention relates to a clean germicidal floor and/or floor mat for areas where people need to take off their shoes and walk for various reasons. A prime example is airport security area where travelers are required to take off their shoes for security check before getting on the airplane. This invention can be further applied to deter and/or stop the spreading or transmission of disease causing or harmful microorganisms or spores thereof from infected individuals to other individuals at sports centers, gyms, locker rooms, public shower area, at home etc. This invention relates to specially designed floor system(s) that uses germicidal ultraviolet lights (UV-C) alone and/or other UV lights such as UV-A and UV-B and/or in combination with germicidal chemicals that will deactivate or render harmless the disease causing germs and/or spores thereof.

More specifically, the present invention relates to devices that are designed to protect individuals from contracting diseases or transmitting diseases through contact through foot and shoes and related thereof. The invention also provides clean germicidal floor and/or floor mat for areas where people need to take off their shoes and walk for various reasons. The invention further provides means to deter and/or stop the spreading or transmission of disease causing or harmful microorganisms or spores thereof from infected individuals to other individuals. Specifically the invention provides devices that use germicidal ultraviolet light incorporated in or on a target floor or mat to kill or deactivate harmful germs (mold, fungi, bacteria, viruses) on the floor or the feet or the socks or related thereof from spreading and causing harms to people or animals that come in contact.

The invention is not only limited to using germicidal ultraviolet lights alone but also chemicals such as antibiotics or chemicals with germicidal properties that in contact with germs will kill or deactivate the microorganisms or spores thereof. Furthermore this invention may be applied in combinations of ultraviolet light and germicidal chemicals thereof.

This invention also provides devices with circulating air that air born germs released in the floor area, where people walk in barefoot or in socks or in shoes or related thereof, are filtered and be deactivated or killed through actions of ultraviolet light and/or in combinations of antibiotics or chemicals with germicidal properties that in contact with germs will kill or deactivate the germs or spores thereof.

When people need to take off their shoes for airport security check, they run into possibilities of transmitting germs from one person to another or several individuals. These germs could be highly toxic mold, fungi, bacteria, viruses and spores thereof. These problems are not only limited to airports but also everywhere else, such as gym, swimming pool areas, public shower, athletic facility locker rooms and shower, etc. where there is a possibility of similar situations where people need to take off their shoes and walk on a common area with foot traffic, either barefoot or with socks on or related thereof.

The invention relates to devices that will kill or deactivate not only one or two germs that cause diseases but an array of all known and yet to be discovered disease causing microorganisms using ultraviolet light (UV-C) alone and/or in combination with germicidal chemicals that could be transmitted through contact through foot and/or socks and/or shoes and/or related thereof.

The disease causing microorganisms is not limited to those deactivated or killed by UV-C alone but also other ultraviolet lights such as UV-A and UV-B or in combinations ultraviolet lights thereof. In addition, the target disease causing germs are not limited to those that are deactivated or killed by UV-C alone but also other ultraviolet lights such as UV-A and UV-B but also those that are further deactivated or killed by germicidal chemicals.

Therefore, one aspect of the present invention is directed to devices that are designed to protect individuals from contracting diseases or transmitting diseases through contact through foot and shoes and related thereof.

One aspect of the invention is to provide protection to people who run into possibilities of transmitting germs from one person to another or several individuals because they need to take off their shoes for various reasons.

One aspect of the invention is to provide protection to people who run into possibilities of transmitting germs from one person to another or several individuals because they need to take off their shoes for airport security check.

One aspect of the invention is to provide protection to people who run into possibilities of transmitting germs from one person to another or several individuals because they need to take off their shoes for everywhere else, such as gym, swimming pool areas, public shower, athletic facility locker rooms and shower, pedicure salon, individual home etc.

One aspect of the invention is to provide protection to people who run into possibilities of transmitting germs from one person to another or several individuals because they need to take off their shoes and walk on a common area with foot traffic, either barefoot or with socks on.

Another aspect of the invention is to provide clean germicidal floor and/or floor mat for areas where people need to take off their shoes and walk for various reasons. Another aspect of the invention provides means to deter and/or stop the spreading or transmission of disease causing or harmful microorganisms or spores thereof from infected individuals to other individuals.

Another aspect of the invention provides devices that use germicidal ultraviolet light incorporated in or on a target floor or mat to kill or deactivate harmful germs (mold, fungi, bacteria, viruses) on the foot or the socks or the floor from spreading and causing harms to people or animals that come in contact.

Another aspect of the invention is not only limited to using germicidal ultraviolet lights alone but also chemicals such as antibiotics or chemicals with germicidal properties that in contact with germs will kill or deactivate the microorganisms or spores thereof.

Another aspect of the invention relates to applications in combinations of ultraviolet light and germicidal chemicals thereof.

Another aspect of the invention provides devices with circulating air that air born germs released in the floor area, where people walk in barefoot or in socks or in shoes or related thereof, are filtered and be deactivated or killed through actions of ultraviolet light and/or in combinations of antibiotics or chemicals with germicidal properties that in contact with germs will kill or deactivate the germs or spores thereof.

One aspect of this invention deals with germs that could be highly toxic mold, fungi, bacteria, viruses and spores thereof.

Another aspect of the invention relates to devices that will kill or deactivate not only one or two germs that cause diseases but an array of all known and yet to be discovered disease causing microorganisms using ultraviolet light (UV-C) alone and/or in combination with germicidal chemicals.

Another aspect of the invention relates to disease causing microorganisms is not limited to those deactivated or killed by UV-C alone but also other ultraviolet lights such as UV-A and UV-B or in combinations of ultraviolet lights thereof.

Another aspect of the invention relates to disease causing germs not limited to those that are deactivated or killed by UV-C alone but also other ultraviolet lights such as UV-A and UV-B and also those that are further deactivated or killed by germicidal chemicals.

One aspect of the present invention is directed to devices that are designed to use various sizes of the ultraviolet light sources.

Another aspect of the invention is directed to devices that are designed to use various numbers, sizes and strength of the ultraviolet light sources.

Another aspect of the invention is directed to devices that are designed to use various numbers and sizes and strength of the ultraviolet light sources.

Another aspect of the invention is directed to devices that are designed to easily replace the ultraviolet light sources when device malfunction occurs.

Another aspect of the invention is directed to devices that are designed to trap dirt and dust through grid floor design.

Another aspect of the invention is directed to devices that are designed to replace or clean the trapped dirt and dust through grid floor design.

Another aspect of the invention is directed to devices that are designed to have airflow through fan and air filter system.

Another aspect of the invention is directed to devices that are designed to easily replace fan and air filter system when needed.

One aspect of the invention is directed to devices that are of different width and length to accommodate the requirement and need to be effective and cost efficient.

Another aspect of the invention is directed to devices that are of different width and length to accommodate the requirement of Department of Homeland Security, athletic facility, pedicure beauty salon, gymnasium, public shower and locker room area, and individual home but not limited to the list.

### DETAILED DESCRIPTION OF THE DRAWINGS

Referring now to the figures attached hereto, FIGS. 1 and 2 is a perspective view of one embodiment of a germicidal floor system 10 (hereinafter referred to as “the system”) constructed in accordance with the principles of the present invention. The system 10 is a free-standing unit capable of being set up in generally any location. The system 10 generally comprises a housing 12, a grate 13, a plurality of ultraviolet light sources 16a-16d, and two means for moving air 18a, 18b.

The housing 12 comprises a base plate 20, a first channel 22a, a second channel 22b, a first support plate 24a, and a second support plate 24b. Each of the channels 22a, 22b of the presently disclosed embodiment include generally arch-shaped cross-sections. In at least one embodiment, the channels 22a, 22b can include light reflectors on the interior surfaces thereof for reflecting light waves generated by the plurality of ultraviolet light sources 16a-16d, as will be described. In one embodiment, the light reflectors can include mirrors, for example.

The grate 13 of the disclosed embodiment comprises a porous surface such as a grate having discrete dimensions and a plurality of openings 25. For the sake of description, the top of the grate 13 includes a target surface 14. The grate 13 can be constructed of generally any material such as plastic, aluminum, steel, etc. The grate 13 is supported by the base plate 20 of the housing 12, between the first and second support plates 24a, 24b. So configured, the housing 12 defines first and second elongated openings 26a, 26b in the first and second channels 22a, 22b, respectively. The first and second elongated openings 26a, 26b are disposed generally adjacent to and on opposite sides of the target surface 14.

In FIGS. 1 and 2, the grate 13 is generally rectangular and, as illustrated, slightly raised above the base plate 20 of the housing 12. This allows for dirt and other debris to pass through and be trapped beneath the grate 13. The grate 13 is therefore preferably removable from the housing 12 such that dirt or other debris can be cleaned from the top of the base plate 20.

Each of the plurality of ultraviolet light sources 16a-16d comprises an elongated bulb, for example, mounted within a ballast (not shown) within the respective first and second channels 22a, 22b. The present embodiment illustrates four (4) ultraviolet light sources 16a-16d, i.e., two (2) light sources in each channel 22a, 22b. Alternative embodiments, however, can have generally any number of ultraviolet light sources 16a-16d mounted within each channel 22a, 22b. When activated, the ultraviolet light sources 16a-16b generate ultraviolet light waves. The shape and configuration of the channels 22a, 22b, as well as the specific orientation of the light sources 16a-16d within the channels 22a, 22b, direct and reflect the light waves out of the elongated openings 26a, 26b and toward the target surface 14 of the grate 13, as is schematically depicted in FIG. 3, for example. So configured, the ultraviolet light waves can kill or deactivate harmful germs or transmissible diseases disposed on the target surface 14 of the grate 13, as well as harmful germs or transmissible diseases carried by the feet, socks, or shoes worn by a person walking across the grate 13.

As mentioned, the system 10 further includes two means for moving air 18a, 18b. Each of the means for moving air 18a, 18b comprises one or more electric fans 28. The fans 28 are disposed between the base plate 20 and the respective first and second support plates 24a, 24b of the housing 12. In one embodiment, the fans 28 can comprise centrifugal fans such that flow within the fans is mainly radial to a shaft of the fans. In another embodiment, the fans 28 can comprise axial fans such that flow within the fans is mainly parallel to a shaft of the fans. Moreover, as illustrated in FIG. 2, the support plates 24a, 24b of the housing 12 include screened portions 30 positioned directly above the fans 28. The screened portions 30 define a plurality of openings 32. So configured, when the fans 28 are in operation, air is drawn vertically from above the system 10 and through the openings 25 in the grate 13. Then, the air is drawn horizontally outwardly between the base plate 20 and the support plates 24a, 24b toward the fans 28. The fans 28, in combination with the structural geometry of the housing 12, then redirect the air upwards through the openings 32 in the support plates 24a, 24b toward the ultraviolet light sources 16a-16d. As such, the air comes into intimate contact with the ultraviolet light sources 16a-16d, thereby allowing the ultraviolet light waves to kill or deactivate any air born harmful germs or transmissible diseases carried by the drawn air. Finally, as the air flow passed the ultraviolet light sources 16a-16d, the shape and configuration of the channels 22a, 22b, i.e., the arch-shape of the channels 22a, 22b, directs the air flow out of the system 10 and back toward the grate 13. This entire flow path is generally illustrated by the plurality of arrows provided on FIG. 2.

FIG. 2 also illustrates a pair of air filters 34a, 34b positioned between the grate 13 and the housing 12. The air filters 34a, 34b can comprise germicidal filters such that the filters 30a, 30b further help kill or deactivate any air born harmful germs or transmissible diseases carried by the drawn air. Each of the air filters 30a, 30b can comprise a single filter or a combination of a plurality of filters arranged along the length of the system 10.

FIG. 4 schematically depicts an alternative embodiment of a germicidal floor system 100 constructed in accordance with the principles of the present invention. Although each of the details are not expressly depicted, the system 100 depicted in FIG. 4 is generally similar to the system 10 described above with reference to FIGS. 1-3, except that the system 100 additionally includes two means for emitting a germicidal chemical 102 into the region of the target surface 14 of the grate 13, as well as optionally onto the target surface 14 of the grate 13. Similar to the means for moving air 18a, 18b described above, the means for emitting a germicidal chemical 102 of the present embodiment is disposed within the channels 22a, 22b of the housing 12 and adjacent opposite sides of the grate 13. The means for emitting a germicidal chemical 102 can comprise vaporizers, misters, or any other type of device capable of emitting a liquid or gaseous substance into the region of the grate 13. Because this potentially introduces a liquid to the system 100, the housing 12 is further equipped with a pair of drains 104 positioned adjacent to the grate 13. The drains 104 are capable of collecting the used chemical. In some embodiments, the germicidal chemical can include an antibiotic chemical, for example.

While the system 10 depicted in FIGS. 1-3 has been described as including a housing 12 with channels 22a, 22b having generally arch-shaped cross-sections, alternative designs are intended to be within the scope of the present invention. FIGS. 5A-5C, for example, illustrate three alternative systems 10 having channels 22a, 22b with alternative cross-sectional designs. Moreover, as depicted in FIG. 5C, it should be appreciated that the channels 22a, 22b can have different cross-sectional designs.

FIG. 6 illustrates a prototype of a germicidal floor system 200 constructed in accordance with the principles of the present invention. The system 200 is constructed of a plurality of system components 202 linearly arranged together to form a long walkway for individuals to walk along. FIG. 7 illustrates one such system component 202, which could be used by itself for individuals to stand on, for example. Each of the system components 202 are constructed in a manner generally identical to one of the systems 10, 100 described above with the exception that each component 202 includes a pair of channels 222a, 222b having a generally square cross-section, which can be seen more clearly in FIG. 7.

The system 200 depicted in FIG. 6 is constructed of four (4) system components 202. However, alternative systems 200 could be constructed of generally any number of system components 202. Therefore, it should be appreciated that the present invention provides a modular-type construction that allows for any length or dimension germicidal floor system to be constructed as desired.

In light of the foregoing, the present disclosure could generally be stated as comprising a floor device and/or devices that are designed to protect individuals from contracting diseases or transmitting diseases through floor contact through foot and shoes and related thereof.

Additionally, the present disclosure could generally be stated as comprising a clean germicidal floor device and/or devices that are designed to protect individuals who run into possibilities of transmitting germs from one person to another or several individuals because they need to take off their shoes and walk on a common area with foot traffic, either barefoot or with socks on that include for airport security check and everywhere else, such as gym, swimming pool areas, public shower, athletic facility locker rooms and shower, pedicure salon, individual home etc.

Additionally, the present disclosure could generally be stated as comprising a floor device and/or devices that are designed to use germicidal ultraviolet light in or on a target floor or mat to kill or deactivate harmful germs (mold, fungi, bacteria, viruses, parasitic worms, etc) on the floor or the feet or the socks from spreading and causing harms to people or animals that come in contact.

Additionally, the present disclosure could generally be stated as comprising a floor device and/or devices that are designed but not only limited to using germicidal ultraviolet lights alone but also chemicals such as antibiotics or chemicals with germicidal properties that in contact with germs will kill or deactivate the microorganisms or spores thereof.

Additionally, the present disclosure could generally be stated as comprising a floor device and/or devices that are designed to circulate air that air born germs released in the floor area, where people walk in barefoot or in socks or in shoes or related thereof, are filtered and be deactivated or killed through actions of ultraviolet light and/or in combinations of antibiotics or chemicals with germicidal properties that in contact with germs will kill or deactivate the germs or spores thereof.

Additionally, the present disclosure could generally be stated as comprising a floor device and/or devices that are designed to kill or deactivate not only one or two germs that can cause diseases but an array of all known and yet to be discovered disease causing microorganisms using ultraviolet light (UV-C) alone and/or in combination with other UV lights such as UV-A and UV-B and/or in combination with germicidal chemicals.

Additionally, the present disclosure could generally be stated as comprising a floor device and/or devices that will protect individuals from transmissible disease causing germs and/or spores thereof through contact through feet using germicidal ultraviolet light (UV-C) alone and/or in combination of other ultraviolet lights such as UV-A and UV-B with germicidal chemicals.

Additionally, the present disclosure could generally be stated as comprising a floor device and/or devices that will protect individuals from transmissible disease causing germs and/or spores thereof through contact through feet using germicidal ultraviolet light (UV-C) alone and/or in combination of other UV lights such as UV-A and UV-B with germicidal chemicals that will irradiate and/or expose the germicidal chemicals to the feet area that will come in contact with the floor.

Additionally, the present disclosure could generally be stated as comprising a floor device and/or devices that will protect individuals from transmissible disease causing germs and/or spores thereof through contact through feet using germicidal ultraviolet light (UV-C) alone and/or in combination of other ultraviolet lights such as UV-A and UV-B with germicidal chemicals and circulating filtered air through ultraviolet light (UV) incorporated germicidal air filter device(s).

Additionally, the present disclosure could generally be stated as comprising a floor device and/or devices that will protect individuals from transmissible disease causing germs and/or spores thereof through contact through feet using germicidal ultraviolet light (UV-C) alone and/or in combination of other UV lights such as UV-A and UV-B with germicidal chemicals that will irradiate and/or expose the germicidal chemicals to the feet area that come in contact with the floor and circulating filtered air through ultraviolet light (UV) incorporated germicidal air filter device(s).

Additionally, the present disclosure could generally be stated as comprising a floor device and/or devices that will protect individuals from transmissible disease causing germs and/or spores thereof through contact through feet using germicidal ultraviolet light (UV-C) alone and/or in combination of other UV lights such as UV-A and UV-B with germicidal chemicals that will irradiate and/or expose the germicidal chemicals to the feet area that will come in contact with the floor; whereas the UV light source(s) are easily replace when in need (state of breakage and/or the light bulb going out)

Additionally, the present disclosure could generally be stated as comprising a method of deterring, stopping, killing or deactivating germs that could spread or transmit disease causing or harmful microorganisms such as mold, fungi, bacteria, viruses, parasites and spores thereof from infected individuals to other individuals through contact through foot, socks, shoes and/or related thereof.

Additionally, the present disclosure could generally be stated as comprising a method of protecting individuals from transmissible disease causing germs and/or spores thereof through contact through feet; wherein the germs include viruses, bacteria, fungi, parasitic worms, mold, etc.

Additionally, the present disclosure could generally be stated as comprising a method of protecting individuals from transmissible disease causing germs and/or spores thereof through contact through feet using germicidal ultraviolet light (UV-C) alone and/or in combination with other UV light such as UV-A and UV-B and in the presence of germicidal chemicals and circulating filtered air through ultraviolet light (UV) incorporated germicidal air filter device(s).

Additionally, the present disclosure could generally be stated as comprising a floor device and/or devices that are designed to use various numbers and sizes and strength (wattage) of the ultraviolet light sources.

Additionally, the present disclosure could generally be stated as comprising a floor device and/or devices that are designed to easily replace the ultraviolet light sources when device malfunction occurs.

Additionally, the present disclosure could generally be stated as comprising a floor device and/or devices that are designed to replace or clean the trapped dirt and dust through grid floor design.

Additionally, the present disclosure could generally be stated as comprising a floor device and/or devices that are designed to have airflow through fan and air filter system that is designed to easily replace fan and air filter system when needed.

Additionally, the present disclosure could generally be stated as comprising a floor device and/or devices that are of different width and length to accommodate the requirement of airport security check protocol of Department of Homeland Security, athletic facility, pedicure beauty salon, gymnasium, public shower and locker room area, and individual home.

This invention is not limited to what is described herein but rather is also inclusive of similar, equivalent, and derivative technology, designs, methods of use, and applications that are obvious to those versed in the art.

What is claimed is:

1. A device, comprising:
a housing;
a target surface of discrete dimensions supported by the housing and adapted to be walked upon by an individual; and
at least one ultraviolet light source supported by the housing and adapted to direct ultraviolet light waves onto the target surface such that the ultraviolet light waves interact with the feet of the individual walking upon the target surface, wherein the ultraviolet light waves kill or deactivate harmful germs or transmissible diseases disposed on the target surface, or carried by the feet or the socks of the individual, thereby preventing the harmful germs from spreading and causing harm to other individuals or animals.

2. The device of claim 1, further comprising a means for moving air through the target surface and adjacent the at least one ultraviolet light source to kill or deactivate air born germs or transmissible diseases released in the area of the target surface.

3. The device of claim 2, further comprising a germicidal filter disposed adjacent to the target surface such that at least some of the air circulated by the air circulation means travels through the germicidal filter.

4. The device of claim 2, wherein the target surface comprises a porous surface and the means for circulating air through the target surface comprises a fan for drawing air downward through the target surface.

5. The device of claim 1, further comprising a means for emitting a germicidal chemical in the area of the target surface to kill or deactivate harmful germs or transmissible diseases.

6. The device of claim 5, wherein the germicidal chemical comprises an antibiotic chemical.

7. The device of claim 1, wherein the ultraviolet light waves comprise at least one of UV-C light waves, UV-B light waves, and UV-A light waves.

8. The device of claim 1, wherein the housing comprises a pair of channels disposed on opposite sides of the target surface, the at least one ultraviolet light source and a light reflector disposed within at least one of the channels.

9. The device of claim 8, wherein the channels include elongated openings adjacent the target surface for directing the ultraviolet light waves toward the target surface.

10. The device of claim 1, wherein the target surface comprises a removable grate to enable the removal of trapped debris from beneath the target surface.

11. A device, comprising:
a housing defining a base plate, and first and second channels extending along opposite sides of the base plate;
a target surface of discrete dimensions supported by the base plate of the housing and adapted to be walked upon by an individual;
a first ultraviolet light source and a light reflector supported by the housing within the first channel; and
a second ultraviolet light source and a light reflector supported by the housing within the second channel,
the first and second channels of the housing shaped and configured with the light reflector to reflect ultraviolet light waves generated by the first and second ultraviolet light sources onto the target surface such that the ultraviolet light waves interact with the feet of the individual walking upon the target surface to kill or deactivate harmful germs or transmissible diseases disposed on the target surface, or carried by the feet or socks of the individual, thereby preventing the harmful germs or transmissible diseases from spreading and causing harm to other individuals or animals.

12. The device of claim 11, wherein the first and second channels of the housing define first and second elongated openings extending the length of the target surface.

13. The device of claim 11, further comprising a means for moving air through the target surface and into the first and second channels adjacent to the first and second ultraviolet light sources to kill or deactivate air born germs or transmissible diseases released in the area of the target surface.

14. The device of claim 13, further comprising a germicidal filter disposed adjacent to the target surface such that at least some of the air circulated by the air circulation means travels through the germicidal filter.

15. The device of claim 13, wherein the target surface comprises a porous surface and the means for circulating air through the target surface comprises a fan for drawing air downward through the target surface.

16. The device of claim 11, further comprising a means for emitting a germicidal chemical in the area of the target surface to kill or deactivate harmful germs.

17. The device of claim 16, wherein the germicidal chemical comprises an antibiotic chemical.

18. The device of claim 11, wherein the ultraviolet light waves comprise at least one of UV-C light waves, UV-B light waves, and UV-A light waves.

19. The device of claim 11, wherein the target surface comprises a removable grate supported by the base plate of the housing to enable the removal of trapped debris from beneath the target surface.

20. A method of killing or deactivating harmful germs or transmissible diseases disposed on a target surface of a cleaning device, or carried by the feet or socks of an individual walking on the target surface, the method comprising:
generating ultraviolet light waves with one or more ultraviolet light sources disposed within channels disposed on opposing sides of the target surface of the cleaning device; and
at least one of the following:
deflecting the ultraviolet light waves out of the channels and toward the target surface such that the ultraviolet light waves interact with at least one of the target surface, the individuals feet walking across the target surface, and the socks worn by the individual walking across the target surface to kill or deactivate harmful germs or transmissible diseases disposed on the target surface or carried by the feet or socks of the individual;
circulating air through the target surface and toward the one or more ultraviolet light sources disposed within the channels to kill or deactivate air born germs or transmissible diseases released in the area of the target surface;
emitting a germicidal chemical in the area of the target surface to kill or deactivate harmful germs or transmissible diseases.
